(19) United States
(12) Patent Application Publication
(10) Pub. No.: US 2021/0155720 A1
(43) Pub. Date: May 27, 2021

(54) Method for Preparing Hyaluronan Odd-numbered Oligosaccharides by Double Enzyme Hydrolysis

(71) Applicant: Jiangnan University, Wuxi (CN)

(72) Inventors: Zhen KANG, Wuxi (CN); Jian CHEN, Wuxi (CN); Guocheng DU, Wuxi (CN); Jing HE, Wuxi (CN); Hao HUANG, Wuxi (CN)

(21) Appl. No.: 17/168,261

(22) Filed: Feb. 5, 2021

Related U.S. Application Data
(63) Continuation of application No. PCT/CN2019/100409, filed on Aug. 13, 2019.

(30) Foreign Application Priority Data
Aug. 14, 2018 (CN) 2018109203014

(51) Int. Cl. C08B 37/08; C08B 37/00

(52) U.S. Cl. CPC C08B 37/0072; C08B 37/0003

(57) ABSTRACT

The disclosure discloses a method for preparing hyaluronan oligosaccharides with odd-numbered degrees of polymerization by hydrolysis, and belongs to the technical field of biological engineering. The disclosure takes macromolecular hyaluronic acid as the substrate, and by controlling the addition amount and reaction time of two hyaluronic acid hydrolases, leech hyaluronidase (LHase) and bovine testicular hyaluronidase (BTH), simultaneously prepares two different structures of hyaluronan oligosaccharides with odd-numbered degrees of polymerization (trisaccharide, pentasaccharide and heptasaccharide). The disclosure provides a new, simple and feasible method for preparing hyaluronan oligosaccharides with odd-numbered degrees of polymerization, thereby laying a foundation for the research on the functions and characteristics of the hyaluronan oligosaccharides with odd-numbered degrees of polymerization.

### TECHNICAL FIELD

The disclosure relates to a method for preparing hyaluronan oligosaccharides with odd-numbered degrees of polymerization, and belongs to the technical field of biological engineering.

### BACKGROUND

Hyaluronic acid (HA) is a viscous polysaccharide (Mr=10⁵-10⁶ Da) of unbranched disaccharide repetitive units formed by connecting D-glucuronic acid (GlcUA) and N-acetylglucosamine (GlcNAc) by β-1,3 glycosidic bonds and by β-1,4 glycosidic bonds. HA is the main component of the intercellular substance of animal tissues and the natural substance with the strongest water holding capacity. Because of its unique properties, HA is widely used in medicine, clinical diagnosis and treatment, cosmetics, and food and health industries. In recent years, it has been discovered that HA of different molecular masses have different biological activities. Hyaluronan oligosaccharides (o-HAs, Mr=10 kDa or less) have immunological activity and the effects of promoting proliferation of vascular endothelial cells and reversing multidrug resistance of tumor cells.

With the increasing application of hyaluronan oligosaccharides (o-HAs), in recent years, attention has been paid to the degradation of HA and the preparation of degradation products around the world. At present, HA degradation includes 3 main methods: physical degradation, chemical degradation and biodegradation. Among them, biodegradation is most widely used because of its mild, non-toxic and harmless conditions. The commonly used method of biodegradation is enzymatic hydrolysis. Among the hydrolases, bovine testicular hyaluronidase (BTH) and leech hyaluronidase (LHase) have the best specificity and the highest hydrolysis efficiency for hyaluronic acid (HA). The bovine testicular hyaluronidase (BTH) hydrolyzes β-1,4 glycosidic bonds in HA to generate even-numbered oligosaccharides with glucosamine as the reducing end; and the leech hyaluronidase (LHase) hydrolyzes β-1,3 glycosidic bonds in HA to generate even-numbered oligosaccharides with glucuronic acid as the reducing end. The two enzymes act on the two different glycosidic bonds in hyaluronic acid respectively to generate two hyaluronan even-numbered oligosaccharide series with different reducing ends.

Since the previous enzymatic preparation of hyaluronan oligosaccharides is mostly single-enzymatic hydrolysis, most of the products are oligosaccharides with even-numbered degrees of polymerization, so the application research in medicine and other aspects also mainly focuses on hyaluronan oligosaccharides with even-numbered degrees of polymerization. As a large class of hyaluronan oligosaccharides, the preparation and functional studies of hyaluronan oligosaccharides with odd-numbered degrees of polymerization (that is, hyaluronan oligosaccharides having odd-numbered degrees of polymerization) are almost blank. Therefore, studying the preparation of oligosaccharides with odd-numbered degrees of polymerization is of great significance for the research and development of the hyaluronan oligosaccharides family and the exploration and application of new functions of hyaluronan oligosaccharides.

### SUMMARY

The technical problem to be solved by the disclosure is to provide a method for preparing hyaluronan oligosaccharides with odd-numbered degrees of polymerization, and in particular a method for preparing hyaluronan oligosaccharides with odd-numbered degrees of polymerization by hydrolysis with two enzymes, bovine testicular hyaluronidase and leech hyaluronidase.

The disclosure provides a method for preparing hyaluronan oligosaccharides with odd-numbered degrees of polymerization, and the specific technical solution includes the following steps:

(1) hydrolysis of hyaluronic acid: taking hyaluronic acid with a molecular weight of 10⁵-10⁶ Da as a substrate, and using two hyaluronic acid hydrolases, leech hyaluronidase (LHase) and bovine testicular hyaluronidase (BTH) to act on the substrate to obtain a mixture of a series of hyaluronan oligosaccharides with different odd-numbered degrees of polymerization;

(2) separation of hyaluronan oligosaccharides with different degrees of polymerization: separating and purifying the hyaluronan oligosaccharides obtained in step (1) by using an ion exchange column filled with gel, wherein the specific operation is as follows: treating the ion exchange column with an equilibrium liquid, performing elution with an eluent, and collecting products according to eluting peaks of target products; concentrating and desalting the products to obtain the hyaluronan oligosaccharides with odd-numbered degrees of polymerization;

wherein, the order of hydrolysis of the two enzymes is not limited.

In one embodiment of the disclosure, the method includes the following steps:

(1) hydrolysis of hyaluronic acid: taking hyaluronic acid with a molecular weight of 10⁵-10⁶ Da as a substrate; first adding leech hyaluronidase LHase for hydrolysis; after inactivating the enzyme, adding bovine testicular hyaluronidase BTH for hydrolysis, inactivating the enzyme to obtain a mixture of a series of hyaluronan oligosaccharides with different odd-numbered degrees of polymerization;

(2) separation of hyaluronan oligosaccharides with different degrees of polymerization: separating and purifying the hyaluronan oligosaccharides obtained in step (1) by using an ion exchange column filled with gel to obtain the hyaluronan oligosaccharides with different odd-numbered degrees of polymerization.

In one embodiment of the disclosure, the method includes the following steps:

(1) hydrolysis of hyaluronic acid: taking hyaluronic acid with a molecular weight of 10⁵-10⁶ Da as a substrate; first adding bovine testicular hyaluronidase BTH for hydrolysis; after inactivating the enzyme, adding leech hyaluronidase LHase for hydrolysis, inactivating the enzyme to obtain a mixture of a series of hyaluronan oligosaccharides with different odd-numbered degrees of polymerization;

(2) separation of hyaluronan oligosaccharides with different degrees of polymerization: separating and purifying the hyaluronan oligosaccharides obtained in step (1) by using an ion exchange column filled with gel to obtain the hyaluronan oligosaccharides with different odd-numbered degrees of polymerization.

In one embodiment of the disclosure, the method includes the following steps:

(1) hydrolysis of hyaluronic acid: taking hyaluronic acid with a molecular weight of 10⁵-10⁶ Da as a substrate; adding bovine testicular hyaluronidase BTH and leech hyaluronidase LHase simultaneously for hydrolysis, inactivating the enzyme to obtain a mixture of a series of hyaluronan oligosaccharides with different odd-numbered degrees of polymerization;

(2) separation of hyaluronan oligosaccharides with different degrees of polymerization: separating and purifying the hyaluronan oligosaccharides obtained in step (1) by using an ion exchange column filled with gel to obtain the hyaluronan oligosaccharides with different odd-numbered degrees of polymerization.

In one embodiment of the disclosure, the separation and purification of the hyaluronan oligosaccharides obtained in step (1) by using the ion exchange column filled with gel include the following specific steps: column packing, equilibration, sample loading, elution, and collection of products according to the product peaks, concentration, desalting and freeze drying, so as to obtain the hyaluronan oligosaccharides with odd-numbered degrees of polymerization.

In one embodiment of the disclosure, the hyaluronic acid reacts with the enzymes in the form of a solution, the concentration of the hyaluronic acid solution is 10-20 mg/mL, and the solvent is water.

In one embodiment of the disclosure, in the step (1), the final concentration of the leech hyaluronidase added is 5000-7000 U/mL, and the final concentration of the bovine testicular hyaluronidase added is 1000-4000 U/mL.

In one embodiment of the disclosure, in the step (1), the action time of the leech hyaluronidase is 1-15 h, and the action time of the bovine testicular hyaluronidase is 1-15 h.

In one embodiment of the disclosure, the ion exchange column filled with gel is an anion exchange column filled with Q Sepharose HP (QHP).

In one embodiment of the disclosure, the equilibrium liquid used for equilibration is a Tris-HCl solution with a pH of 7-9, and the eluent used for elution is an NaCl solution with a concentration of 50-300 mM prepared from the equilibrium liquid.

In one embodiment of the disclosure, the elution volume is 2-20 times the column volume, preferably 10-20 times.

In one embodiment of the disclosure, the elution process adopts linear elution, and the linear elution is performed by first using an eluent with a concentration of 0, and using eluents with the concentration gradually increased to the corresponding concentrations (NaCl solutions with a concentration of 50-300 mM prepared from the equilibrium liquid).

In one embodiment of the disclosure, the desalting method is either a molecular exclusion gel column or a dialysis bag desalting method.

In one embodiment of the disclosure, the molecular exclusion gel column is a Superdex 30 Increase 10/300 GL gel column, and the specification of the dialysis bag is 0.5-1.0 KD.

In one embodiment of the disclosure, the method mainly includes the following steps:

(1) Hydrolysis of Hyaluronic Acid

taking a hyaluronic acid aqueous solution with a concentration of 5-20 mg/mL and a molecular weight of 10⁵-10⁶ Da as a substrate; in the hydrolysis process, first adding leech hyaluronidase (LHase) with a final concentration of 5000-7000 U/mL for hydrolysis for 1-10 h; after inactivating the enzyme, adding bovine testicular hyaluronidase (BTH) with a final concentration of 1000-4000 U/mL for hydrolysis for 1-10 h, inactivating the enzyme by boiling, and performing filtering with a 0.22 μm filter membrane;

(2) Separation of Hyaluronan Oligosaccharides

separating the hyaluronan oligosaccharides from filtrate obtained by filtering using the 0.22 μm filter membrane in step (1) by an ion exchange column, wherein the equilibrium liquid is a Tris-HCl buffer solution with a pH of 6-8, the eluent is an NaCl solution with a concentration of 50-300 mM prepared from the equilibrium liquid, and the specific operation is as follows: packing the column with Q Sepharose HP packing, and performing equilibration with the equilibrium liquid, wherein the flow rate of the equilibrium liquid is 2-5 mL/min; then, loading and eluting a sample, wherein the elution strategy is linear elution with NaCl solutions with a concentration of 0 to 50-300 mM, the elution volume is 10-20 times the column volume, and the flow rate of the eluent is 2-6 mL/min; collecting and concentrating the product, desalting the product through a molecular exclusion gel column or a dialysis bag, and performing freeze drying to obtain the hyaluronan oligosaccharides with odd-numbered degrees of polymerization in the product.

In one embodiment of the disclosure, the method mainly includes the following steps:

(1) Hydrolysis of Hyaluronic Acid

taking a hyaluronic acid aqueous solution with a concentration of 5-20 mg/mL and a molecular weight of 10⁵-10⁶ Da as a substrate; in the hydrolysis process, first adding bovine testicular hyaluronidase (BTH) with a final concentration of 1000-4000 U/mL for hydrolysis for 1-10 h; after inactivating the enzyme, adding leech hyaluronidase (LHase) with a final concentration of 5000-7000 U/mL for hydrolysis for 1-10 h, inactivating the enzyme by boiling, and performing filtering with a 0.22 μm filter membrane;

(2) Separation of Hyaluronan Oligosaccharides

separating the hyaluronan oligosaccharides from filtrate obtained by filtering using the 0.22 μm filter membrane in step (1) by an ion exchange column, wherein the equilibrium liquid is a Tris-HCl buffer solution with a pH of 6-8, the eluent is an NaCl solution with a concentration of 50-300 mM prepared from the equilibrium liquid, and the specific operation is as follows: packing the column with Q Sepharose HP packing, and performing equilibration with the equilibrium liquid, wherein the flow rate of the equilibrium liquid is 2-5 mL/min; then, loading and eluting a sample, wherein the elution strategy is linear elution with NaCl solutions with a concentration of 0 to 50-300 mM, the elution volume is 10-20 times the column volume, and the flow rate of the eluent is 2-6 mL/min; collecting and concentrating the product, desalting the product through a molecular exclusion gel column or a dialysis bag, and performing freeze drying to obtain the hyaluronan oligosaccharides with odd-numbered degrees of polymerization in the product.

In one embodiment of the disclosure, the method mainly includes the following steps:

(1) Hydrolysis of Hyaluronic Acid

taking a hyaluronic acid aqueous solution with a concentration of 5-20 mg/mL and a molecular weight of 10⁵-10⁶ Da as a substrate; in the hydrolysis process, adding bovine testicular hyaluronidase (BTH) with a final concentration of 1000-4000 U/mL and leech hyaluronidase (LHase) with a final concentration of 5000-7000 U/mL simultaneously for hydrolysis for 1-10 h, inactivating the enzyme by boiling, and performing filtering with a 0.22 μm filter membrane;

(2) Separation of Hyaluronan Oligosaccharides

separating the hyaluronan oligosaccharides from filtrate obtained by filtering using the 0.22 μm filter membrane in step (1) by an ion exchange column, wherein the equilibrium liquid is a Tris-HCl buffer solution with a pH of 6-8, the eluent is an NaCl solution with a concentration of 50-300 mM prepared from the equilibrium liquid, and the specific operation is as follows: packing the column with Q Sepharose HP packing, and performing equilibration with the equilibrium liquid, wherein the flow rate of the equilibrium liquid is 2-5 mL/min; then, loading and eluting a sample, wherein the elution strategy is linear elution with NaCl solutions with a concentration of 0 to 50-300 mM, the elution volume is 10-20 times the column volume, and the flow rate of the eluent is 2-6 mL/min; collecting and concentrating the product, desalting the product through a molecular exclusion gel column or a dialysis bag, and performing freeze drying to obtain the hyaluronan oligosaccharides with odd-numbered degrees of polymerization in the product.

The disclosure further provides hyaluronan oligosaccharides with odd-numbered degrees of polymerization, and the structure of the hyaluronan oligosaccharides with odd-numbered degrees of polymerization is as follows:

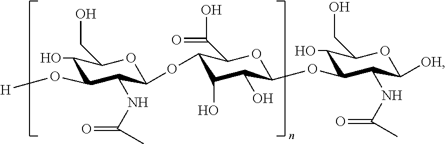

wherein n=1, 2, 3.

In one embodiment of the disclosure, the hyaluronan oligosaccharides with odd-numbered degrees of polymerization are prepared by any of the above methods for preparing the hyaluronan oligosaccharides with odd-numbered degrees of polymerization.

Beneficial Effects of the Disclosure:

(1) Two types of odd-numbered hyaluronan oligosaccharides (type A and type N, with the reducing ends of GlcUA and GlcNAc respectively) can be prepared at the same time by using a new preparation method that uses two hydrolases to act successively. The reaction conditions are mild and the reaction process is safe. By controlling the reaction time and the addition amount of enzymes, preparation of the hyaluronan oligosaccharides with the target odd-numbered degree of polymerization can be achieved. The prepared product is relatively pure.

(2) The new N-type odd-numbered hyaluronan oligosaccharides prepared by the disclosure not only enrich the diversity of oligosaccharide structures, but also are of great significance to develop the relationship between hyaluronan oligosaccharides of different structural types and the occurrence and development of diseases.

### BRIEF DESCRIPTION OF FIGURES

FIG. 1: LCMS ion chromatogram of the hydrolysis product obtained in Example 1.

FIG. 2: LCMS ion chromatogram of the hydrolysis product obtained in Example 2.

FIG. 3: LCMS ion chromatogram of the hydrolysis product obtained in Example 3.

FIG. 4: LCMS ion chromatogram of the hydrolysis product obtained in Example 4.

FIG. 5: mass spectrums of target products 3N saccharide (HA₃^{NN}) in the hydrolysates.

FIG. 6: mass spectrums of target products 3A saccharide (HA₃^{AA}) in the hydrolysates.

FIG. 7: mass spectrums of target products 5N saccharide (HA₅^{NN}) in the hydrolysates.

FIG. 8: mass spectrums of target products 5A saccharide (HA₅^{AA}) in the hydrolysates.

FIG. 9: mass spectrums of target products 7N saccharide (HA₇^{NN}) in the hydrolysates.

FIG. 10: mass spectrums of target products 7A saccharide (HA₇^{AA}) in the hydrolysates.

FIG. 11: molecular structure diagrams of odd-numbered hyaluronan oligosaccharides 3N saccharide (HA₃^{NN}).

FIG. 12: molecular structure diagrams of odd-numbered hyaluronan oligosaccharides 3A saccharide (HA₃^{AA}).

FIG. 13: molecular structure diagrams of odd-numbered hyaluronan oligosaccharides 5N saccharide (HA₅^{NN}).

FIG. 14: molecular structure diagrams of odd-numbered hyaluronan oligosaccharides 5A saccharide (HA₅^{AA}).

FIG. 15: molecular structure diagrams of odd-numbered hyaluronan oligosaccharides 7N saccharide (HA₇^{NN}).

FIG. 16: molecular structure diagrams of odd-numbered hyaluronan oligosaccharides 7A saccharide (HA₇^{AA}).

FIG. 17: general formulas of N-type odd-numbered hyaluronan oligosaccharides and A-type odd-numbered hyaluronan oligosaccharides.

FIG. 18: anion exchange column separation of hyaluronan disaccharide, 3A saccharide (HA₃^{AA}), 3N saccharide (HA₃^{NN}), tetrasaccharide (HA4), 5A saccharide (HA₅^{AA}), 5N saccharide (HA₅^{NN}), and hexasaccharide (HA6).

FIG. 19: anion exchange column separation of hyaluronan 5A saccharide (HA₅^{AA}), 5N saccharide (HA₅^{NN}), hexasaccharide, 7A saccharide (HA₇^{AA}), 7N saccharide (HA₇^{NN}), and octasaccharide (HA8).

### DETAILED DESCRIPTION

The leech hyaluronidase was obtained by recombinantly expressing the leech hyaluronidase gene from the genetically engineered strain of Pichia pastoris by the laboratory, and the nucleotide sequence is shown in SEQ ID NO.1. The bovine testicular hyaluronidase was purchased from Sigma-Aldrich (Shanghai) Trading Co., Ltd.

In the separation and preparation method of mixed oligosaccharides in the hydrolysis product, the chromatography column is an anion exchange column HiTrap QHP (20 mL); the mobile phase is Tris-HCl (pH=8.0); the eluent is 0-300 mmol/L NaCl; the flow rate is 3 mL/min; the detection wavelength is 210 nm. The molecular exclusion gel column is a Superdex 30 Increase 10/300 GL column (with a column size of 10 mm×300-310 mm); the column volume is 24 mL; the column efficiency is greater than 43000 N/m; the typical pressure drop of a packed bed is 3.0 MPa; and the column hardware pressure limit is 5.0 MPa.

The detection method of product purity uses the carbazole sulfuric acid method.

### Example 1: Preparation 1 of Odd-Numbered Hyaluronan 3A Saccharide (HA₃^{AA}), 3N Saccharide (HA₃^{NN}), 5A Saccharide (HA₅^{AA}), and 5N Saccharide (HA₅^{NN})

(1) Hydrolysis of Macromolecular Hyaluronic Acid

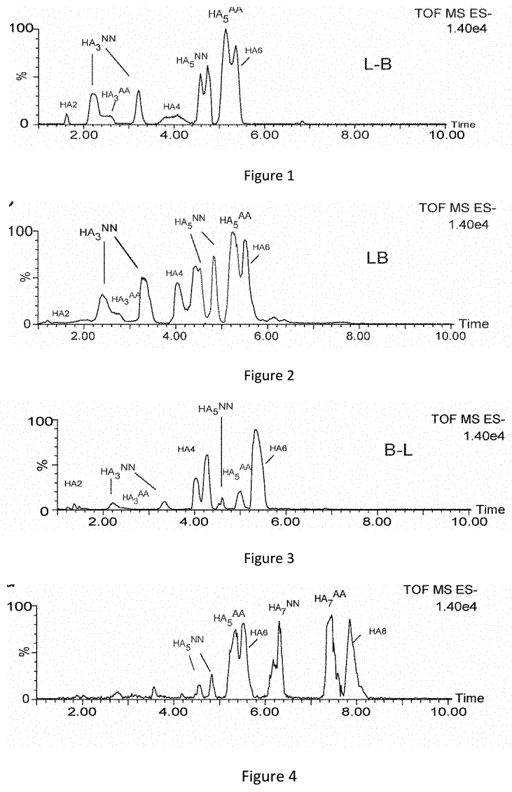

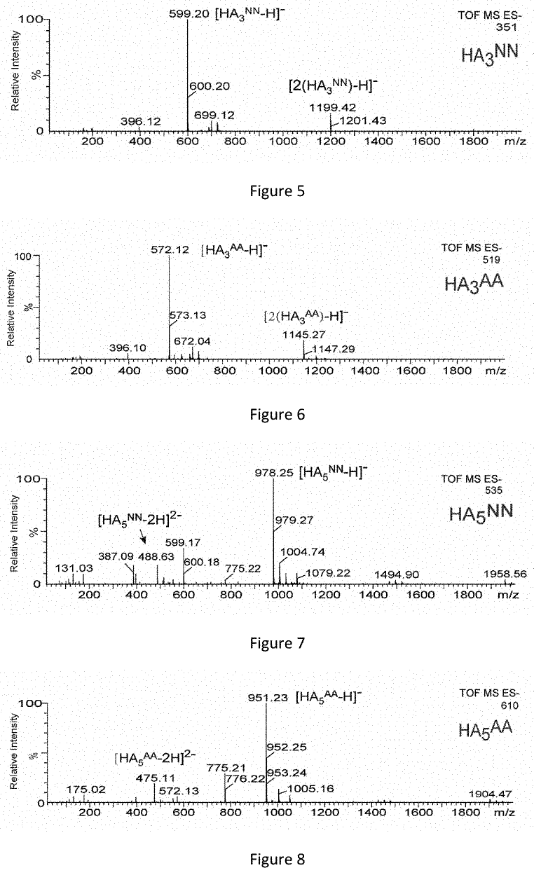

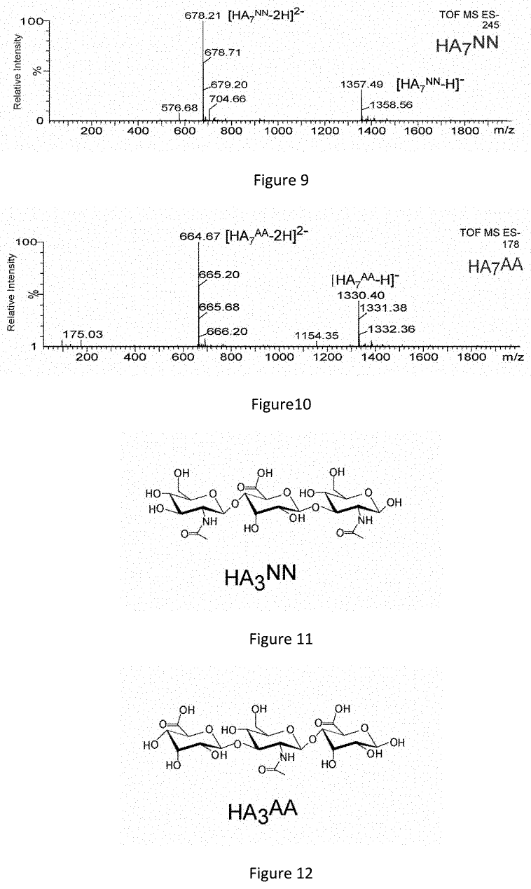

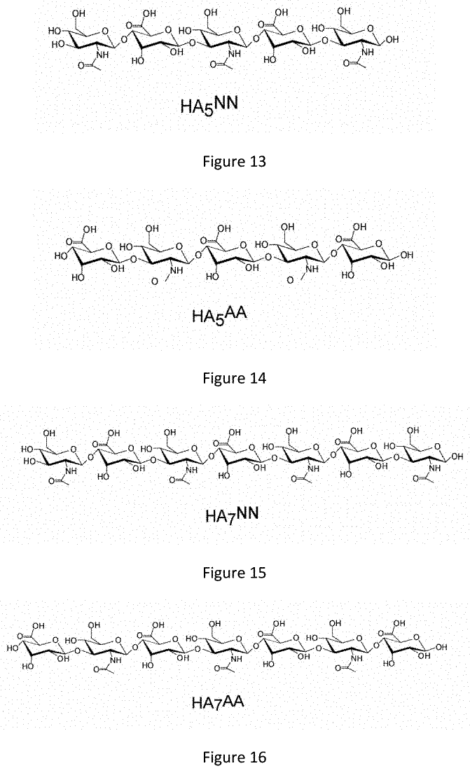

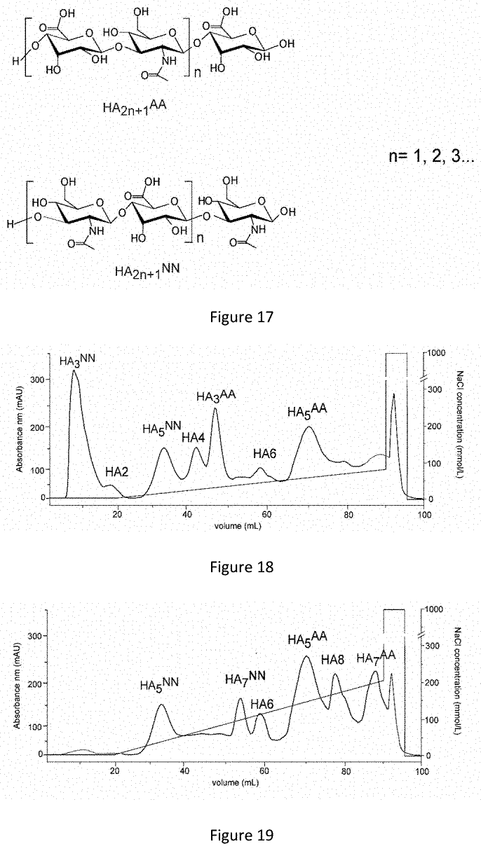

Macromolecular hyaluronic acid powder with a molecular weight of 10⁵-10⁶ Da was dissolved in pure water at a concentration of 10 mg/mL, and fully dissolved overnight. In the hydrolysis process, first leech hyaluronidase LHase was added (the final enzyme concentration was 5000 U/mL). Hydrolysis was performed at 37° C. for 10 hours, and the hydrolysate was boiled at 100° C. to inactivate the enzyme to terminate the reaction. This is the first step of the hydrolysis reaction. After the enzyme in the hydrolysate was inactivated, bovine testicular hyaluronidase BTH with a final concentration of 4000 U/mL was added for hydrolysis at 37° C. for 10 hours, and the enzyme was inactivated at 100° C. by boiling. Impurities were removed, the hydrolysis products were analyzed and identified by using an ultra-high performance liquid chromatography-tandem quadrupole time-of-flight mass spectrometer (MALDI SYNAPT MS) (the results are shown in FIG. 1a and FIGS. 2a, b, c, and d), and the molecular structure diagrams of the hydrolysis products were analyzed and inferred (FIG. 3). By analysis and identification, the hydrolysis products were mainly odd-numbered saccharides including 3A saccharide (HA₃^{AA}), 3N saccharide (HA₃^{NN}), 5A saccharide (HA₅^{AA}), 5N saccharide (HA₅^{NN}) and even-numbered saccharides including disaccharide (HA2), tetrasaccharide (HA4) and hexasaccharide (HA6).

(2) Separation of Hyaluronan Oligosaccharides

A solution A (equilibrium solution), that is, a 50 mM Tris-HCl buffer solution with a pH of 8 was prepared; and a solution B (elution solution), that is, a 80 mM NaCl buffer solution was prepared from the solution A. The separation of hyaluronan oligosaccharides was performed under the following conditions: an HiTrap QHP (5 mL) ion exchange column (with a height-diameter ratio of 3:1) was equilibrated with 1 column volume of solution A at a flow rate of 3 mL/min. The sample was loaded at a flow rate of 1 mL/min. The unbound sample was washed with the solution A, and 3 column volumes were eluted at a flow rate of 3 mL/min. Linear elution was performed with the solution B (first the solution B with a concentration of 0 was used, and the NaCl solution B with the concentration gradually increased to 80 mM prepared from the solution A was used for elution). The elution volume was 1 column volume, and the flow rate was 3 mL/min. The obtained peaks were collected sequentially. The collected peaks were concentrated to obtain sample solutions, and then the sample solutions were desalted by a Superdex 30 Increase 10/300 GL gel column. The products were detected by MALDI SYNAPT MS. The saccharides collected by the ion column were sequentially: 3N saccharide, disaccharide (HA2), 5N saccharide, tetrasaccharide (HA4), 3A saccharide, hexasaccharide (HA6), and 5A saccharide, as shown in FIG. 4a. Finally, the sample was freeze-dried to obtain the odd-numbered saccharides 3A saccharide (HA₃^{AA}), 3N saccharide (HA₃^{NN}), 5A saccharide (HA₅^{AA}), and 5N saccharide (HA₅^{NN}). The yield of the oligosaccharides was measured by the sulfuric acid carbazole method. The purity of the oligosaccharides measured by the sulfuric acid carbazole method is 75%-90%, all of which reach high purity.

### Example 2: Preparation 2 of Odd-Numbered Hyaluronan 3A Saccharide (HA₃^{AA}), 3N Saccharide (HA₃^{NN}), 5A Saccharide (HA₅^{AA}), and 5N Saccharide (HA₅^{NN})

(1) Hydrolysis of Macromolecular Hyaluronic Acid

Macromolecular hyaluronic acid powder with a molecular weight of 10⁵-10⁶ Da was dissolved in pure water at a concentration of 10 mg/mL, and fully dissolved overnight. In the hydrolysis process, BTH with a final concentration of 4000 U/mL and LHase with a final concentration of 5000 U/mL were added simultaneously, hydrolysis was performed at 37° C. for 10 hours, and the hydrolysate was boiled at 100° C. to inactivate the enzyme to terminate the reaction. Impurities were removed, and the hydrolysis products were analyzed and identified by using an ultra-high performance liquid chromatography-tandem quadrupole time-of-flight mass spectrometer (MALDI SYNAPT MS) (as shown in FIG. 1b and FIGS. 2a, b, c, and d). By analysis and identification, the hydrolysis products were mainly odd-numbered saccharides including 3A saccharide (HA₃^{AA}), 3N saccharide (HA₃^{NN}), 5A saccharide (HA₅^{AA}), 5N saccharide (HA₅^{NN}) and even-numbered saccharides including disaccharide (HA2), tetrasaccharide (HA4) and hexasaccharide (HA6). It can be seen from the peak size that the yield of the odd-numbered oligosaccharides in the present example is different from that in Example 1.

(2) Separation of Hyaluronan Oligosaccharides with Different Degrees of Polymerization

The separation steps were the same as in Example 1. Finally, the sample was freeze-dried to obtain the odd-numbered saccharides including 3A saccharide (HA₃^{AA}), 3N saccharide (HA₃^{NN}), 5A saccharide (HA₅^{AA}), and 5N saccharide (HA₅^{NN}). The purity of the oligosaccharides measured by the sulfuric acid carbazole method is 75%-90%, all of which reach high purity.

### Example 3: Preparation 3 of Odd-Numbered Hyaluronan 3A Saccharide (HA₃^{AA}), 3N Saccharide (HA₃^{NN}), 5A Saccharide (HA₅^{AA}), and 5N Saccharide (HA₅^{NN})

(1) Hydrolysis of Macromolecular Hyaluronic Acid

In the present example, the order of hydrolysis of the two enzymes was changed, that is, first the bovine testicular hyaluronidase (BTH) and then the leech hyaluronidase were used for hydrolysis. The rest of the operation steps and conditions were the same as in Example 1. The prepared product was analyzed by using an ultra-high performance liquid chromatography-tandem quadrupole time-of-flight mass spectrometer (MALDI SYNAPT MS). The ion current chromatogram (IC) is shown in FIG. 1c and FIGS. 2a, b, c, and d. By analysis and identification, the hydrolysis products were mainly odd-numbered saccharides including 3A saccharide (HA₃^{AA}), 3N saccharide (HA₃^{NN}), 5A saccharide (HA₅^{AA}), 5N saccharide (HA₅^{NN}) and even-numbered saccharides including disaccharide (HA2), tetrasaccharide (HA4) and hexasaccharide (HA6). It can be seen from the peak size that the yield of the odd-numbered oligosaccharides in the present example is different from those in Examples 1 and 2.

(2) Separation of Hyaluronan Oligosaccharides

The separation steps were the same as in Example 1. Finally, the sample was freeze-dried to obtain the odd-numbered saccharides including 3A saccharide (HA₃^{AA}), 3N saccharide (HA₃^{NN}), 5A saccharide (HA₅^{AA}), and 5N saccharide (HA₅^{NN}). The purity of the oligosaccharides measured by the sulfuric acid carbazole method is 75%-90%, all of which reach high purity.

### Example 4: Preparation of Odd-Numbered Hyaluronan 5A Saccharide (HA₅^{AA}), 5N Saccharide (HA₅^{NN}), 7A Saccharide (HA₇^{AA}), and 7N Saccharide (HA₇^{NN})

(1) Hydrolysis of Macromolecular Hyaluronic Acid

Macromolecular hyaluronic acid powder with a molecular weight of 10⁵-10⁶ Da was dissolved in pure water at a concentration of 10 mg/mL, and fully dissolved overnight. In the hydrolysis process, first LHase was added (the final enzyme concentration was 5000 U/mL). Hydrolysis was performed at 37° C. for 6 hours, and the hydrolysate was boiled at 100° C. to inactivate the enzyme to terminate the reaction. This is the first step of the hydrolysis reaction. After the enzyme in the hydrolysate was inactivated, BTH with a final concentration of 4000 U/mL was added for hydrolysis at 37° C. for 6 hours, and the enzyme was inactivated at 100° C. by boiling. Impurities were removed, the hydrolysis products were analyzed and identified by using an ultra-high performance liquid chromatography-tandem quadrupole time-of-flight mass spectrometer (MALDI SYNAPT MS) (as shown in FIG. 1d, and FIGS. 2c, d, e, and f), and the molecular structure diagrams of the hydrolysis products were analyzed and inferred (FIG. 3). The hydrolysis products were mainly odd-numbered saccharides including 5A saccharide (HA₅^{AA}), 5N saccharide (HA₅^{NN}), 7A saccharide (HA₇^{AA}), 7N saccharide (HA₇^{NN}) and even-numbered saccharides including hexasaccharide (HA6) and octasaccharide (HA8).

(2) Separation of Hyaluronan Oligosaccharides

A solution A (equilibrium solution), that is, a Tris-HCl buffer solution with a pH of 8 and a concentration of 50 mM was prepared; and a solution B (elution solution), that is, a 200 mM NaCl buffer solution was prepared from the solution A. The separation of hyaluronan oligosaccharides was performed under the following conditions: an HiTrap QHP (20 mL) ion exchange column (with a height-diameter ratio of 3:1) was equilibrated with 1 column volume of solution A at a flow rate of 3 mL/min. The sample was loaded at a flow rate of 1 mL/min. 1 column volume was equilibrated with the solution A at a flow rate of 3 mL/min. Linear elution was performed with the solution B, so that the concentration of NaCl in the eluent was 200 mM, the elution volume was 3 column volumes, and the flow rate was 3 mL/min. The obtained peaks were collected sequentially. The collected peaks were concentrated to obtain sample solutions, and then the sample solutions were desalted by a Superdex 30 Increase 10/300 GL gel column. The products were detected by MALDI SYNAPT MS. By identification, the collected peaks were respectively 5N saccharide (HA₅^{NN}), 7N saccharide (HA₇^{NN}), hexasaccharide, 5A saccharide (HA₅^{AA}), octasaccharide, and 7A saccharide (HA₇^{AA}), as shown in FIG. 4b. The purity of the oligosaccharides measured by the sulfuric acid carbazole method is 70%-85%, all of which reach high purity.

### Example 5: Preparation of Odd-Numbered Hyaluronan 5A Saccharide (HA₅^{AA}), 5N Saccharide (HA₅^{NN}), 7A Saccharide (HA₇^{AA}), and 7N Saccharide (HA₇^{NN})

In the present example, the order of hydrolysis of the two enzymes was changed, that is, first the LHase and then the BTH were used for hydrolysis. The rest of the operation steps and conditions were the same as in Example 4. Also, the odd-numbered saccharides including 5A saccharide (HA₅^{AA}), 5N saccharide (HA₅^{NN}), 7A saccharide (HA₇^{AA}), and 7N saccharide (HA₇^{NN}) were prepared. The purity of the oligosaccharides measured by the sulfuric acid carbazole method is 70%-85%, all of which reach high purity.

### Example 6: Preparation of Odd-Numbered Hyaluronan 5A Saccharide (HA₅^{AA}), 5N Saccharide (HA₅^{NN}), 7A Saccharide (HA₇^{AA}), and 7N Saccharide (HA₇^{NN})

In the present example, the order of hydrolysis of the two enzymes was changed, that is, the LHase and the BTH were used for hydrolysis simultaneously. The rest of the operation steps and conditions were the same as in Example 4. Also, the odd-numbered saccharides including 5A saccharide (HA₅^{AA}), 5N saccharide (HA₅^{NN}), 7A saccharide (HA₇^{AA}), and 7N saccharide (HA₇^{NN}) were prepared. The purity of the oligosaccharides measured by the sulfuric acid carbazole method is 70%-85%, all of which reach high purity.

### Example 7

Macromolecular hyaluronic acid powder with a molecular weight of 10⁵-10⁶ Da was dissolved in pure water at a concentration of 20 mg/mL, and fully dissolved overnight. In the hydrolysis process, first leech hyaluronidase LHase was added (the final enzyme concentration was 7000 U/mL). Hydrolysis was performed at 37° C. for 3 hours, and the hydrolysate was boiled at 100° C. to inactivate the enzyme to terminate the reaction. This is the first step of the hydrolysis reaction. After the enzyme in the hydrolysate was inactivated, bovine testicular hyaluronidase BTH with a final concentration of 1000 U/mL was added for hydrolysis for 15 hours, and the enzyme was inactivated at 100° C. by boiling. Impurities were removed, and by analysis and identification, the hydrolysis products were mainly odd-numbered saccharides including 3A saccharide (HA₃^{AA}), 3N saccharide (HA₃^{NN}), 5A saccharide (HA₅^{AA}), 5N saccharide (HA₅^{NN}) and even-numbered saccharides including disaccharide, tetrasaccharide and hexasaccharide.

(2) Separation of Hyaluronan Oligosaccharides

The separation steps were the same as in Example 1. Finally, the sample was freeze-dried to obtain the odd-numbered saccharides including 3A saccharide (HA₃^{AA}), 3N saccharide (HA₃^{NN}), 5A saccharide (HA₅^{AA}), and 5N saccharide (HA₅^{NN}).

### Comparative Example 1: Effect of Concentration of Hyaluronic Acid

Macromolecular hyaluronic acid powder with a molecular weight of 10⁵-10⁶ Da was dissolved in pure water at a concentration of 2 mg/mL, and the rest of the operation steps and conditions were the same as in Example 1. The hydrolysis products were analyzed and identified by using an ultra-high performance liquid chromatography-tandem quadrupole time-of-flight mass spectrometer (MALDI SYNAPT MS). Odd-numbered hyaluronan oligosaccharides could be detected, but the content was too low and the preparation efficiency was low.

### Comparative Example 2: Effect of Concentration of Hyaluronic Acid

Macromolecular hyaluronic acid powder with a molecular weight of 10⁵-10⁶ Da was dissolved in pure water at a concentration of 50 mg/mL, and the rest of the operation steps and conditions were the same as in Example 1. The hydrolysis products were analyzed and identified by using an ultra-high performance liquid chromatography-tandem quadrupole time-of-flight mass spectrometer (MALDI SYNAPT MS). The substrate was not effectively hydrolyzed and no odd-numbered hyaluronan oligosaccharide was obtained.

Although the disclosure has been disclosed as above in preferred examples, it is not intended to limit the disclosure. Anyone familiar with the technology can make various changes and modifications without departing from the spirit and scope of the disclosure.

Therefore, the protection scope of the disclosure should be defined by the claims.

What is claimed is:

1. A method for preparing hyaluronan oligosaccharides with odd-numbered degrees of polymerization, comprising the following steps:
(1) hydrolysis of hyaluronic acid: using two hyaluronic acid hydrolases, leech hyaluronidase LHase and bovine testicular hyaluronidase BTH, to separately act on hyaluronic acid with a molecular weight of 10⁵-10⁶ Da to obtain a mixture of hyaluronan oligosaccharides with different odd-numbered degrees of polymerization; and
(2) separation of hyaluronan oligosaccharides with different degrees of polymerization: separating and purifying the mixture of hyaluronan oligosaccharides with different odd-numbered degrees of polymerization obtained in step (1) by using an ion exchange column filled with gel, wherein the separating and purifying comprises: treating the ion exchange column with an equilibrium liquid, performing elution with an eluent, and collecting products according to eluting peaks of target products; concentrating and desalting the products to obtain the hyaluronan oligosaccharides with odd-numbered degrees of polymerization, and
wherein an order of hydrolysis of two enzymes is not limited.

2. The method of claim 1, wherein the hyaluronic acid reacts with the enzymes in a form of a solution, a concentration of the hyaluronic acid solution is 10-20 mg/mL, and a solvent is water.

3. The method of claim 2, wherein in the step (1), a final concentration of the leech hyaluronidase added is 5000-7000 U/mL, and the action time is 1-15 h; and a final concentration of the bovine testicular hyaluronidase added is 1000-4000 U/mL, and the action time is 1-15 h.

4. The method of claim 1, wherein a desalting method in step (2) is either a gel column or a dialysis bag desalting method.

5. The method of claim 1, wherein in step (2), the ion exchange column filled with gel is an anion exchange column filled with Q Sepharose HP.

6. The method of claim 1, wherein in step (2), the equilibrium liquid is a Tris-HCl solution with a pH of 7-9, and the eluent is an NaCl solution with a concentration of 50-300 mM prepared from the equilibrium liquid.

7. The method of claim 1, wherein in step (2), an elution process adopts linear elution, and an elution volume is 10-20 times a column volume.

8. The method of claim 1, wherein the hydrolysis of the hyaluronic acid comprises the following steps:
taking a hyaluronic acid aqueous solution with a concentration of 5-20 mg/mL and a molecular weight of 10⁵-10⁶ Da as a substrate; in a hydrolysis process, first adding leech hyaluronidase with a final concentration of 5000-7000 U/mL for hydrolysis for 1-10 h; after inactivating an enzyme, adding bovine testicular hyaluronidase with a final concentration of 1000-4000 U/mL for hydrolysis for 1-10 h, inactivating an enzyme by boiling, and performing filtering with a 0.22 μm filter membrane; and
the separation of the hyaluronan oligosaccharides comprises the following steps:
separating the hyaluronan oligosaccharides from filtrate obtained by filtering using the 0.22 μm filter membrane in step (1) by an ion exchange column, wherein the equilibrium liquid is a Tris-HCl buffer solution with a pH of 6-8, the eluent is an NaCl solution with a concentration of 50-300 mM prepared from the equilibrium liquid, and the separating the hyaluronan oligosaccharides comprises: packing the column with Q Sepharose HP packing, and performing equilibration with the equilibrium liquid, wherein a flow rate of the equilibrium liquid is 2-5 mL/min; then, loading and eluting a sample, wherein an elution strategy is linear elution, an elution volume is 10-20 times a column volume, and a flow rate of the eluent is 2-6 mL/min; collecting and concentrating a product, desalting the product through a gel column, and performing freeze drying to obtain the hyaluronan oligosaccharides with odd-numbered degrees of polymerization in the product.

9. The method of claim 8, wherein an order of using the two enzymes in the step (1) are exchanged.
